(19) United States
(12) Patent Application Publication
(10) Pub. No.: US 2016/0243012 A1
(43) Pub. Date: Aug. 25, 2016

(54) SKIN COSMETIC

(71) Applicant: KAO CORPORATION, Tokyo (JP)

(72) Inventor: Tomokazu YOSHIDA, Suginami-ku (JP)

(73) Assignee: KAO CORPORATION, Tokyo (JP)

(21) Appl. No.: 15/027,341

(22) PCT Filed: Apr. 10, 2015
(86) PCT No.: PCT/JP15/61219

§ 371 Date: Apr. 5, 2016

(30) Foreign Application Priority Data
May 30, 2014 (JP) 2014-113235

(51) Int. Cl. A61K 8/43; A61Q 19/00; A61K 8/19; A61K 8/34; A61K 8/36; A61K 8/41

(52) U.S. Cl. CPC A61K 8/43; A61K 8/361; A61K 8/41; A61K 8/19; A61K 8/342; A61K 8/345; A61Q 19/007; A61K 2800/52

(57) ABSTRACT

The present invention provides a skin cosmetic containing the following components (A), (B), (C), (D), (E), and (F):

(A) 0.5 to 8 mass % of a linear saturated fatty acid having 12 to 22 of carbon atom,
(B) 0.05 to 5 mass % of an organic base,
(C) 0.01 to 2 mass % of an inorganic base,
(D) 0.1 to 5 mass % of a linear saturated alcohol having 12 to 22 of carbon atom,
(E) 0.1 to 20 mass % of a moisturizing component, and
(F) water, wherein
the total amount of the components (A) and (D) is (A)+(D)=from 0.6 to 13 mass %, the mass ratio of the component (A) to the total amount of the components (A) and (D) [(A)+(D)] is (A)/[(A)+(D)]=from 0.7 to 0.9, the molar ratio of the component (B) to the total amount of the components (B) and (C) [(B)+(C)] is (B)/[(B)+(C)]=from 60 to 90 mol %, and the molar ratio of the total amount of the components (B) and (C) [(B)+(C)] to the component (A) is [(B)+(C)]/(A)=from 20 to 100 mol %.

### TECHNICAL FIELD

The present invention relates to a skin cosmetic.

### BACKGROUND ART

Cosmetics supplemented with moisturizers such as polyols (e.g., glycerin), various oil agents, amino acids, and hyaluronic acid have been developed for the purpose of protecting the skin from external environments such as dryness or physical stimulation or moisturizing the skin, thereby softening the skin and keeping the youthful appearance of the skin. These cosmetics protect the skin from external stimulation and also prevent water loss from the inside of the skin through the application of an emulsified layer containing an oil and water onto the skin, while contributing to improvement in rough skin or dry skin by imparting a given amount of moisture to the skin.

In contrast, cosmetics containing horny layer intercellular lipid components such as ceramides and further having a lamellar structure similar to intercellular lipids have been proposed (Non Patent Literature 1 and Patent Literature 1).

Meanwhile, a technique using fatty acid arginine salt is known as a technique of forming a lamellar structure without the use of ceramides, etc. (Non Patent Literature 2).

Further studies have been made on cosmetics containing fatty acid arginine salt combined with a higher alcohol or a surfactant (e.g., Patent Literature 2).
(Non Patent Literature 1) The journal of investigative dermatology, vol. 96 (6), 845-851 (1991)
(Non Patent Literature 2) J. Oleo. Sci., vol. 54 (6), 325-333 (2005)
(Patent Literature 1) JP-A-H6-345633
(Patent Literature 2) JP-A-2007-9199

### SUMMARY OF INVENTION

The present invention relates to a skin cosmetic containing the following components (A), (B), (C), (D), (E), and (F):

(A) 0.5 to 8 mass % of a linear saturated fatty acid having 12 to 22 of carbon atom,
(B) 0.05 to 5 mass % of an organic base,
(C) 0.01 to 2 mass % of an inorganic base,
(D) 0.1 to 5 mass % of a linear saturated alcohol having 12 to 22 of carbon atom,
(E) 0.1 to 20 mass % of a moisturizing component, and
(F) water, wherein
the total amount of the components (A) and (D) is (A)+(D)=from 0.6 to 13 mass %, the mass ratio of the component (A) to the total amount of the components (A) and (D) [(A)+(D)] is (A)/[(A)+(D)]=from 0.7 to 0.9, the molar ratio of the component (B) to the total amount of the components (B) and (C) [(B)+(C)] is (B)/[(B)+(C)]=from 60 to 90 mol %, and the molar ratio of the total amount of the components (B) and (C) [(B)+(C)] to the component (A) is [(B)+(C)]/(A)=from 20 to 100 mol %.

### DETAILED DESCRIPTION OF INVENTION

When amphipathic solid fats having a high melting point, such as ceramides, are contained in oil-in-water emulsions, the separation, gelation, or the like of the emulsions over time tends to occur and makes these solid fats difficult to contain stably. Hence, there are high limitations on production methods or formulation.

Since ceramides are easily crystallized, a method using a large amount of an emulsifier for ceramides has been proposed. The problem of such preparations, however, is poor spreadability over the skin upon application.

In addition, ceramides themselves are expensive and therefore present problems associated with versatility.

The techniques using fatty acid arginine salt without the use of ceramides as described in Non Patent Literature 2 and Patent Literature 4 are insufficient in terms of the stability of an oil agent, moisturizing properties, etc.

The present invention relates to a skin cosmetic which forms a cosmetic coating film having a lamellar structure on the surface of the skin after being applied to the skin and thereby, allows a moisturizing component to uniformly adhere onto the skin for a long time, offers no stickiness derived from the moisturizing component, has a high moisturizing effect, and sustains the effect.

The present inventor has found that a lamellar membrane is formed and a large amount of a moisturizing component can be stably retained in the lamellar membrane, by combining a particular saturated fatty acid, a saturated alcohol, an organic base, an inorganic base, a moisturizing component, and water at a particular ratio; thus, the obtained skin cosmetic allows a moisturizing component to uniformly adhere onto the skin for a long time, offers no stickiness derived from the moisturizing component, has a high moisturizing effect, and sustains the effect.

The skin cosmetic of the present invention has a lamellar structure where, in a lamella formed as a continuous phase (hereinafter, referred to as a bulk lamella) in the cosmetic, a large amount of moisture or a moisturizing component is present between the layers of the formed lamella; hence, water or the moisturizing component retained in the lamellar membrane remains on the skin without being diffused even after application to the skin. A major feature of a coating film having a lamellar structure formed on the skin is to maintain the structure for a long time without being crystallized even after water evaporation.

Meanwhile, in most of conventional cosmetics, water or a moisturizing component is evaporated or diffused after application to the skin and therefore hardly remains on the skin for a long time. Thus, the structure and properties of such a lamella seem to contribute to the excellent effects of the present invention in terms of moisture-confining properties, the sustention of a moist feeling, the absence of stickiness, uniform distribution of moisture on the skin, etc.

In general, the presence of the lamellar structure can be confirmed by conducting small-angle X-ray diffractometry and observing repeated diffraction peaks characteristic of the lamellar structure in the obtained X-ray diffraction profile. In the present invention, the bulk lamella can be confirmed under a polarization microscope (sample thickness: 25 mm). Specifically, an object found shiny as a whole in the visual field indicates the presence of the bulk lamella.

The skin cosmetic of the present invention has a stable lamellar structure because of its excellent preservation stability. The skin cosmetic of the present invention can retain a moisturizing component in the lamellar membrane and therefore has a high moisturizing effect and sustains the effect for a long time. Furthermore, the moisturizing component retained in the lamellar membrane can uniformly adhere onto the skin for a long time. Because even a large amount of a moisturizing component, when contained therein, is covered with the lamellar membrane, the skin cosmetic of the present invention achieves less perceivable stickiness derived from the moisturizing component.

### (A) Linear Saturated Fatty Acid Having 12 to 22 of Carbon Atom:

The component (A) used in the present invention is a linear saturated fatty acid having 12 to 22 of carbon atom, and examples thereof include lauric acid, myristic acid, palmitic acid, stearic acid, and behenic acid. Among them, a linear saturated fatty acid having 14 to 22 of carbon atom is preferred, and a linear saturated fatty acid having 16 to 22 of carbon atom is more preferred. At least any one selected from the group consisting of palmitic acid and stearic acid is further preferred from the viewpoint of forming a stable lamellar structure in the cosmetic and from the viewpoint of forming a bulk lamella having a large amount of moisture or a moisturizing component between the lamella layers as the lamella. Stearic acid is still further preferred from the viewpoint of the stability of the lamellar structure.

At least one or two or more selected from the group consisting of these linear saturated fatty acids having 12 to 22 of carbon atom can be used as the component (A) and can form a lamellar structure in the cosmetic by neutralization with the components (B) and (C) mentioned later. Thus, the component (A) is present as a fatty acid or a salt thereof in the cosmetic. In the present invention, the content of the component (A) is an amount in terms of the fatty acid.

The content of the component (A) in the whole composition is 0.5 mass % or more, preferably 1.5 mass % or more, more preferably 2.5 mass % or more, and is 8 mass % or less, preferably 7 mass % or less, more preferably 6 mass % or less, from the viewpoint of forming a stable lamellar structure in the cosmetic and from the viewpoint of forming a bulk lamella and a concentric lamella as the lamella. The content of the component (A) in the whole composition is from 0.5 to 8 mass %, preferably from 1.5 to 7 mass %, more preferably from 2.5 to 6 mass %.

### (B) Organic Base:

The component (B) used in the present invention is an organic base, and examples thereof include alkylamines having an alkyl group having 1 to 6 of carbon atom, alkanolamines having an alkyl group having 1 to 6 of carbon atom, and basic amino acids. The component (B) functions as a neutralizing agent for the component (A).

Specifically, examples of the alkylamines include methylamine, ethylamine, propylamine, butylamine, hexylamine, dimethylamine, and diethylamine. Examples of the alkanolamines include monoethanolamine, diethanolamine, triethanolamine, N-methyl-diethanolamine, N,N-dimethyl monoethanolamine, and aminomethyl propanol. Aminomethyl propanol is preferred. Examples of the basic amino acids include lysine, histidine, and arginine. Arginine is preferred. The arginine is preferably L-arginine.

Among them, an alkanolamine having an alkyl group having 1 to 6 of carbon atom or a basic amino acid having 1 to 6 of carbon atom is preferred, an alkanolamine having an alkyl group having 3 to 6 of carbon atom or a basic amino acid having 3 to 6 of carbon atom is more preferred, a basic amino acid is further preferred, aminomethyl propanol or arginine is still further preferred, and arginine is still further preferred, from the viewpoint of neutralizing the component (A) and forming a stable lamellar structure in the cosmetic, from the viewpoint of forming a bulk lamella having a large amount of moisture or a moisturizing component between the lamella layers as the lamella, from the viewpoint of suppressing the deposition of crystals, and from the viewpoint of enhancing moisturizing properties. The arginine is preferably L-arginine.

At least one or two or more selected from the group consisting of these organic bases can be used as the component (B). The molar ratio of the component (B) to the component (A) is preferably 10 mol % or more, more preferably 30 mol % or more, and is preferably 80 mol % or less, more preferably 60 mol % or less, from the viewpoint of enhancing the stability of the lamellar structure in the skin cosmetic, from the viewpoint of suppressing the deposition of crystals, and from the viewpoint of enhancing moisturizing properties.

The content of the component (B) in the whole composition is 0.05 mass % or more, preferably 0.15 mass % or more, more preferably 0.5 mass % or more, and is 5 mass % or less, preferably 4 mass % or less, more preferably 3.5 mass % or less, from the viewpoint of enhancing the stability of the lamellar structure in the skin cosmetic and enhancing moisturizing properties. The content of the component (B) in the whole composition is from 0.05 to 5 mass %, preferably from 0.15 to 4 mass %, more preferably from 0.5 to 3.5 mass %.

The component (B) may form salts with the component (A) and other acids in the cosmetic. In the present invention, the content of the component (B) is an amount in terms of the organic base.

### (C) Inorganic Base:

The component (C) used in the present invention is an inorganic base, and examples thereof include alkali metal hydroxides such as sodium hydroxide and potassium hydroxide. The component (C) functions as a neutralizing agent for the component (A).

Among them, sodium hydroxide or potassium hydroxide is preferred, and sodium hydroxide is more preferred, from the viewpoint of neutralizing the component (A), from the viewpoint of forming a stable lamellar structure in the cosmetic, and from the viewpoint of forming a bulk lamella having a large amount of moisture or a moisturizing component between the lamella layers as the lamella.

One or two or more of these inorganic bases can be used as the component (C). The molar ratio of the component (C) to the component (A) is preferably 10 mol % or more, more preferably 30 mol % or more, and is preferably 80 mol % or less, more preferably 60 mol % or less, from the viewpoint of enhancing the stability of the lamellar structure in the skin cosmetic and enhancing moisturizing properties.

The content of the component (C) in the whole composition is 0.01 mass % or more, preferably 0.05 mass % or more, more preferably 0.1 mass % or more, and is 2 mass % or less, preferably 0.5 mass % or less, more preferably 0.3 mass % or less, from the viewpoint of enhancing the stability of the lamellar structure in the skin cosmetic and enhancing moisturizing properties. The content of the component (C) in the whole composition is from 0.01 to 2 mass %, preferably from 0.05 to 0.5 mass %, more preferably from 0.1 to 0.3 mass %.

The component (C) may form salts with the component (A) and other acids in the cosmetic. In the present invention, the content of the component (C) is an amount in terms of the inorganic base.

In the present invention, the molar ratio of the component (B) to the total amount of the components (B) and (C) [(B)+(C)], (B)/[(B)+(C)], is (B)/[(B)+(C)]=from 60 to 90 mol %, preferably from 65 to 85 mol %, more preferably from 70 to 80 mol %, from the viewpoint of suppressing crystallization, from the viewpoint of forming a film excellent in moisture-confining properties, and from the viewpoint of suppressing sliminess.

The molar ratio of the total amount of the components (B) and (C), [(B)+(C))] to the component (A), [(B)+(C)]/(A), represents the degree of neutralization and is [(B)+(C)]/(A)=from 20 to 100 mol %, preferably from 50 to 95 mol %, more preferably from 60 to 90 mol %, from the viewpoint of forming two types of lamellar structures, i.e., a bulk lamella and a concentric lamella, from the viewpoint of forming a film excellent in moisture-confining properties, from the viewpoint of suppressing crystallization, and from the viewpoint of suppressing foaming.

The combination of the component (B) and the component (C) is preferably aminomethyl propanol or arginine as the component (B) and sodium hydroxide or potassium hydroxide as the component (C), more preferably arginine as the component (B) and sodium hydroxide as the component (C), from a similar viewpoint.

### (D) Linear Saturated Alcohol Having 12 to 22 of Carbon Atom:

The component (D) used in the present invention is a linear saturated alcohol having 12 to 22 of carbon atom, and examples thereof include lauryl alcohol, myristyl alcohol, cetyl alcohol, stearyl alcohol, and behenyl alcohol. Among them, a linear saturated alcohol having 14 to 22 of carbon atom is preferred, and a linear saturated alcohol having 16 to 22 of carbon atom is more preferred. Cetyl alcohol or stearyl alcohol is further preferred, and still further preferably, the component (D) contains cetyl alcohol and stearyl alcohol, from the viewpoint of stabilizing the lamellar structure formed by the component (A) in the cosmetic and from the viewpoint of forming a bulk lamella and a concentric lamella as the lamella. When the component (D) contains cetyl alcohol and stearyl alcohol, these alcohols may be contained each independently or as a mixture, or cetostearyl alcohol, which is a mixture thereof, may be contained.

At least one or two or more selected from the group consisting of these linear saturated alcohols having 12 to 22 of carbon atom can be used as the component (D). The content thereof in the whole composition is 0.1 mass % or more, preferably 0.5 mass % or more, more preferably 1 mass % or more, and is 5 mass % or less, preferably 3 mass % or less, more preferably 2.5 mass % or less, from the viewpoint of stabilizing the lamellar structure in the cosmetic and from the viewpoint of forming a bulk lamella having a large amount of moisture or a moisturizing component between the lamella layers as the lamella. The content of the component (D) in the whole composition is from 0.1 to 5 mass %, preferably from 0.5 to 3 mass %, more preferably from 1 to 2.5 mass %.

In the present invention, the total amount of the components (A) and (D), (A)+(D), in the whole composition is 0.6 mass % or more, preferably 2 mass % or more, more preferably 4.5 mass % or more, and is 13 mass % or less, preferably 10 mass % or less, more preferably 8 mass % or less, from the viewpoint of enhancing the preservation stability of the cosmetic composition. Also, the total amount of the components (A) and (D) is (A)+(D)=from 0.6 to 13 mass %, preferably from 2 to 10 mass %, more preferably from 4.5 to 8 mass %.

The mass ratio of the component (A) to the total amount of the components (A) and (D) [(A)+(D)], (A)/[(A)+(D)], is 0.7 or more, preferably 0.73 or more, more preferably 0.75 or more, and is 0.9 or less, preferably 0.87 or less, more preferably 0.82 or less, from the viewpoint of forming a lamella in the cosmetic composition while suppressing crystallization and from the viewpoint of enhancing moisturizing properties. The mass ratio of the component (A) to the total amount of the components (A) and (D) [(A)+(D)] is (A)/[(A)+(D)]=from 0.7 to 0.9, preferably from 0.73 to 0.87, more preferably from 0.75 to 0.82.

### (E) Moisturizing Component:

The moisturizing component as the component (E) used in the present invention can be any moisturizing component for use in usual cosmetics. The moisturizing component is preferably a polyhydric alcohol, more preferably a dihydric or trihydric polyhydric alcohol, from the viewpoint of improving moisturizing properties.

Examples of the dihydric or trihydric polyhydric alcohol include ethylene glycol, diethylene glycol, polyethylene glycol (number-average molecular weight: smaller than 1000), propylene glycol, 1,3-propanediol, dipropylene glycol, polypropylene glycol (number-average molecular weight: smaller than 1000), 1,3-butylene glycol, glycerin, diglycerin, polyglycerin, and isoprene glycol.

The moisturizing component as the component (E) has a molecular weight of preferably from 70 to 600, more preferably from 75 to 500, further preferably from 80 to 300, still further preferably from 85 to 200, from the viewpoint of improving moisturizing properties.

The component (E) is at least any one selected from the group consisting of preferably propylene glycol, 1,3-propanediol, dipropylene glycol, 1,3-butylene glycol, glycerin, and diglycerin, more preferably 1,3-propanediol, 1,3-butylene glycol, dipropylene glycol, and glycerin.

At least one or two or more of these moisturizing components can be used as the component (E).

The content of the component (E) in the whole composition is 0.1 mass % or more, preferably 1 mass % or more, more preferably 2.5 mass % or more, and is 20 mass % or less, preferably 18 mass % or less, more preferably 15 mass % or less, from the viewpoint of improving moisturizing properties. The content of the component (E) in the whole composition is from 0.1 to 20 mass %, preferably from 1 to 18 mass %, more preferably from 2.5 to 15 mass %.

In the present invention, the mass ratio of the component (E) to the total amount of the components (A) and (D) [(A)+(D)], (E)/[(A)+(D)], is preferably 0.1 or more, more preferably 0.15 or more, further preferably 0.3 or more, and is preferably 3 or less, more preferably 2.5 or less, further preferably 1.5 or less, from the viewpoint of moisturizing properties, the absence of stickiness, and the uniformity of application on the skin. Also, the mass ratio of the component (E) to the total amount of the components (A) and (D) [(A)+(D)], (E)/[(A)+(D)], is preferably from 0.1 to 3, more preferably from 0.15 to 2.5, further preferably from 0.3 to 1.5.

### (F) Water:

The component (F) used in the present invention is water and serves as a solvent for the cosmetic of the present invention. The component (F) becomes a balance of the components. The components (A) to (F) can be combined to form a stable lamellar α-gel structure in the cosmetic.

The content of the component (F) in the whole composition is preferably 50 mass % or more, more preferably 60 mass % or more, and is preferably 95 mass % or less, more preferably 90 mass % or less, from the viewpoint of forming a stable lamellar structure in the cosmetic and from the viewpoint of forming a bulk lamella having a large amount of moisture or a moisturizing component between the lamella layers as the lamella. The content of the component (F) in the whole composition is preferably from 50 to 95 mass %, more preferably from 60 to 90 mass %.

The skin cosmetic of the present invention containing the components (A) to (F) forms a lamellar structure. This cosmetic is applied to the skin to form a cosmetic coating film on the surface of the skin as a result of water evaporation. This cosmetic coating film is a film formed by a bulk lamella having a large amount of moisture or a moisturizing component between the lamella layers. A coating film having a lamellar structure formed on the skin maintains the structure for a long time without being crystallized even after water evaporation and allows water or the moisturizing component remained in the lamella layers to remain on the skin without being diffused. As a result, water loss from the skin can be suppressed, while moisture can be imparted to the skin, thereby sustaining high moisturizing properties. Since the moisturizing component, which is a main cause of stickiness, is retained between the lamella layers constituting the film, the film is excellent in the absence of stickiness. A further feature of the cosmetic of the present invention is to have the high uniformity of application of moisture or the moisturizing component on the skin. Although a detailed reason therefor is unclear, this is probably because the bulk lamella retaining water or the moisturizing component is uniformly spread over the irregularities of the skin, such as the sulci cutis, the cristae cutis, or texture, and the resulting film has high adhesion to the skin.

### (G) Nonionic Surfactant:

In the present invention, the skin cosmetic can further contain a nonionic surfactant from the viewpoint of facilitating forming the lamellar structure as described above and improving the stability of the lamellar structure, and from the viewpoint of suppressing foaming.

The nonionic surfactant can also prevent the skin from becoming white due to fine foams which are generated due to the friction between the skin and fingers when the skin cosmetic of the present invention is applied to the skin by fingers. As a result, the cosmetic can be well spread over the skin when applied thereto.

Examples of the nonionic surfactant include: ethylene glycol fatty acid esters such as ethylene glycol monostearic acid ester; polyethylene glycol fatty acid esters such as polyethylene glycol (2) monostearic acid ester; polyalkylene glycol alkyl ethers such as polyethylene glycol (5) decyl pentadecyl ether; polyethylene glycol hydrogenated castor oils such as polyethylene glycol (5) hydrogenated castor oil monoisolaurate; propylene glycol fatty acid esters; monoglycerin monofatty acid esters such as glycerin monoisostearic acid ester; monoglycerin difatty acid esters such as glycerin distearic acid ester and glycerin dilauric acid ester; glycerin alkyl ethers such as glycerin monoisostearyl ether; sorbitan fatty acid esters such as sorbitan monostearic acid ester; fatty acid alkanolamides; and fatty acid dialkanolamides such as lauric acid diethanolamide.

Among these nonionic surfactants, a polyalkylene glycol alkyl ether having an alkyl group having 12 to 22 of carbon atom is preferred, and a polyalkylene glycol ether having an alkyl group having 12 to 18 of carbon atom is more preferred, from the viewpoint of suppressing fine foams upon application.

At least one or two or more selected from the group consisting of these nonionic surfactants can be used as the nonionic surfactant as the component (G). The component (G) preferably contains at least two polyalkylene glycol ethers differing in the number of carbon atom in their alkyl groups by 2 or more, more preferably contains at least two selected from the group consisting of polyethylene glycol ether of lauryl alcohol (laureth-3), polyethylene glycol ether of cetyl alcohol, and polyethylene glycol ether of stearyl alcohol, and further preferably contains these 3 polyethylene glycol ethers, from the aforementioned viewpoint as well as from the viewpoint of enhancing moisturizing properties and from the viewpoint of suppressing sliminess. In the case of using two or more polyalkylene glycol ethers having an alkyl group having 12 to 18 of carbon atom, these polyalkylene glycol ethers may be contained each independently or as a mixture, or ceteareth-20, which is a mixture of polyethylene glycol ether of cetyl alcohol and polyethylene glycol ether of stearyl alcohol, may be contained. Still further preferably, the component (G) contains laureth-3 and ceteareth-20.

The content of the nonionic surfactant as the component (G) in the whole composition is preferably 0.1 mass % or more, more preferably 0.3 mass % or more, further preferably 0.5 mass % or more, and is preferably 2 mass % or less, more preferably 1.8 mass % or less, further preferably 1.5 mass % or less, from the viewpoint of improving moisturizing properties, from the viewpoint of suppressing sliminess, from the viewpoint of improving stability, and from the viewpoint of suppressing the foaming of the cosmetic composition upon application. The content of the component (G) in the whole composition is preferably from 0.1 to 2 mass %, more preferably from 0.3 to 1.8 mass %, further preferably from 0.5 to 1.5 mass %.

The mass ratio of the total amount of the components (A) and (D) [(A)+(D)] to the component (G), [(A)+(D)]/G, is preferably 3 or more, more preferably 4 or more, further preferably 5 or more, and is preferably 30 or less, more preferably 20 or less, and further preferably 15 or less, from the viewpoint of improving moisturizing properties, from the viewpoint of suppressing sliminess, from the viewpoint of improving stability, and from the viewpoint of suppressing the foaming of the cosmetic composition. The mass ratio of the total amount of the components (A) and (D) [(A)+(D)] to the component (G), [(A)+(D)]/G, is preferably from 3 to 30, more preferably from 4 to 20, further preferably from 5 to 15.

The skin cosmetic of the present invention can further contain, appropriately, for example, an oil component other than those described above, a surfactant other than those described above, a thickener, a microbicide, a wetting agent, a colorant, an antiseptic, a feel-improving agent, a powder, a fragrance, an anti-inflammatory agent, a whitening agent, an antiperspirant, an ultraviolet absorber, and an antioxidant as components for use in usual cosmetics, in addition to the component (A) to the component (G).

The skin cosmetic of the present invention can further contain a solid fat other than the components (A) and (D). The content thereof is preferably 10 mass % or less, more preferably 8 mass % or less, further preferably 5 mass % or less, still further preferably 3 mass % or less, from the viewpoint of stabilizing the lamellar structure.

In the present invention, the skin cosmetic can further contain an anionic surfactant, from the viewpoint of improving the stability of the lamellar structure, from the viewpoint of improving moisturizing properties, from the viewpoint of reducing an oily feel, from the viewpoint of improving the sustention of gloss, and from the viewpoint of suppressing foaming.

The anionic surfactant is selected from the group consisting of anionic surfactants except for the component (A), and examples thereof include: alkyl sulfuric acid esters having 12 to 22 of carbon atom or salts thereof, such as sodium lauryl sulfate and potassium lauryl sulfate; alkyl ether sulfuric acid esters having 12 to 22 of carbon atom or salts thereof, such as polyoxyethylene lauryl sulfate triethanolamine; N-acyl sarcosines having 12 to 22 of carbon atom or salts thereof, such as sodium lauroyl sarcosine; alkyl phosphates having 12 to 22 of carbon atom or salts thereof, such as sodium monostearyl phosphate; alkyl ether phosphates having 12 to 22 of carbon atom of polyoxyethylene or salts thereof, such as sodium polyoxyethylene oleyl ether phosphate and sodium polyoxyethylene stearyl ether phosphate; dialkyl sulfosuccinates having 12 to 24 of carbon atom or salts thereof, such as sodium di-2-ethylhexyl sulfosuccinate; N-alkyloyl methyltaurines having 12 to 22 of carbon atom or salts thereof, such as sodium N-stearoyl-N-methyltaurine; and N-acyl glutamates having 12 to 22 of carbon atom or salts thereof, such as sodium dilauroyl glutamate, monosodium N-lauroyl glutamate, sodium N-stearoyl-L-glutamate, arginine N-stearoyl-L-glutamate, sodium N-stearoyl glutamate, and sodium N-myristoyl-L-glutamate.

At least one or more selected from the group consisting of these anionic surfactants can be used as the anionic surfactant, and one of these anionic surfactants can be used alone, or two or more thereof can be used in combination.

The content of the anionic surfactant in the whole composition is preferably 0.01 mass % or more, more preferably 0.1 mass % or more, further preferably 0.3 mass % or more, and is preferably 2 mass % or less, more preferably 1.8 mass % or less, further preferably 1.5 mass % or less, from the viewpoint of improving the stability of the lamellar structure, from the viewpoint of improving moisturizing properties, from the viewpoint of reducing an oily feel, from the viewpoint of improving the sustention of gloss, and from the viewpoint of suppressing foaming.

The skin cosmetic of the present invention can be produced, for example, by: mixing the components (B), (C), (E), and (F) and homogeneously dissolving the mixture at 60 to 100° C. to form an aqueous phase portion; mixing the components (A) and (D) and homogeneously dissolving the mixture at 60 to 100° C. to form an oil phase portion; and homogeneously mixing the aqueous phase portion with the oil phase portion and cooling the mixture to preferably from 5 to 30° C., more preferably from 10 to 28° C., further preferably from 15 to 26° C.

The skin cosmetic of the present invention has a pH of preferably from 5.5 to 9.5, more preferably from 6.0 to 9.0, further preferably from 6.5 to 8.5, at 25° C. from the viewpoint of forming a stable lamellar structure in the cosmetic. In the present invention, the pH is determined by directly measuring the pH of a sample at 25° C. using a pH meter (F-52, manufactured by Horiba, Ltd.).

The skin cosmetic of the present invention can be prepared as, for example, a skin lotion, an emulsion, a cream, a gel, or a serum and is particularly preferably used as a cream or a gel. The skin cosmetic of the present invention can be used as a sheet-shaped cosmetic in which a sheet-shaped base material such as woven cloth or nonwoven cloth is impregnated with the skin cosmetic of the present invention or the skin cosmetic of the present invention is applied to such a base material.

The skin cosmetic of the present invention can be applied, for use, to the skin, preferably the skin except for the scalp, more preferably any of the face, the body, limbs, and the like.

The skin cosmetic of the present invention can moisturize the skin by application to the skin.

As for the aforementioned embodiments, the present invention further discloses the following compositions and methods for using the same and methods for producing the same.

<1> A skin cosmetic containing the following components (A), (B), (C), (D), (E), and (F):
(A) 0.5 to 8 mass % of a linear saturated fatty acid having 12 to 22 of carbon atom,
(B) 0.05 to 5 mass % of an organic base,
(C) 0.01 to 2 mass % of an inorganic base,
(D) 0.1 to 5 mass % of a linear saturated alcohol having 12 to 22 of carbon atom,
(E) 0.1 to 20 mass % of a moisturizing component, and
(F) water, wherein
the total amount of the components (A) and (D) is (A)+(D)=from 0.6 to 13 mass %, the mass ratio of the component (A) to the total amount of the components (A) and (D) [(A)+(D)] is (A)/[(A)+(D)]=from 0.7 to 0.9, the molar ratio of the component (B) to the total amount of the components (B) and (C) [(B)+(C)] is (B)/[(B)+(C)]=from 60 to 90 mol %, and the molar ratio of the total amount of the components (B) and (C) [(B)+(C)] to the component (A) is [(B)+(C)]/(A)=from 20 to 100 mol %.
<2> The skin cosmetic according to <1>, wherein the component (A) is preferably a linear saturated fatty acid having 14 to 22 of carbon atom, more preferably a linear saturated fatty acid having 16 to 22 of carbon atom.
<3> The skin cosmetic according to <1> or <2>, wherein the component (A) is preferably at least any one selected from the group consisting of lauric acid, myristic acid, palmitic acid, stearic acid, and behenic acid, more preferably at least any one selected from the group consisting of palmitic acid and stearic acid.
<4> The skin cosmetic according to any one of <1> to <3>, wherein the content of the component (A) in the whole composition is preferably 1.5 mass % or more, more preferably 2.5 mass % or more, and is preferably 7 mass % or less, more preferably 6 mass % or less.
<5> The skin cosmetic according to any one of <1> to <4>, wherein the component (B) preferably contains at least any one or more of alkylamines selected from the group consisting of methylamine, ethylamine, propylamine, butylamine, hexylamine, dimethylamine, and diethylamine; alkanolamines selected from the group consisting of monoethanolamine, diethanolamine, triethanolamine, N-methyl-diethanolamine, N,N-dimethyl monoethanolamine, and aminomethyl propanol; and basic amino acids selected from the group consisting of lysine, histidine, and arginine.
<6> The skin cosmetic according to any one of <1> to <5>, wherein the component (B) is preferably an alkanolamine having an alkyl group having 1 to 6 of carbon atom or a basic amino acid having 1 to 6 of carbon atom, more preferably an alkanolamine having an alkyl group having 3 to 6 of carbon atom or a basic amino acid having 3 to 6 of carbon atom, further preferably a basic amino acid.
<7> The skin cosmetic according to any one of <1> to <6>, wherein the component (B) is preferably aminomethyl propanol or arginine, more preferably arginine, wherein the arginine is preferably L-arginine.
<8> The skin cosmetic according to any one of <1> to <7>, wherein the content of the component (B) in the whole composition is preferably 0.15 mass % or more, more preferably 0.5 mass % or more, and is preferably 4 mass % or less, more preferably 3.5 mass % or less.
<9> The skin cosmetic according to any one of <1> to <8>, wherein the component (C) is preferably sodium hydroxide or potassium hydroxide.
<10> The skin cosmetic according to any one of <1> to <9>, wherein the content of the component (C) in the whole composition is preferably 0.05 mass % or more, more preferably 0.1 mass % or more, and is preferably 0.5 mass % or less, more preferably 0.3 mass % or less.
<11> The skin cosmetic according to any one of <1> to <10>, wherein the molar ratio of the component (C) to the total amount of the components (B) and (C) [(B)+(C)], (B)/[(B)+(C)], is preferably (B)/[(B)+(C)]=from 65 to 85 mol %, more preferably from 70 to 80 mol %.
<12> The skin cosmetic according to any one of <1> to <11>, wherein the molar ratio of the total amount of the components (B) and (C) [(B)+(C))] to the component (A), [(B)+(C)]/(A), is preferably [(B)+(C)]/(A)=from 50 to 95 mol %, more preferably from 60 to 90 mol %.
<13> The skin cosmetic according to any one of <1> to <12>, wherein the combination of the component (B) and the component (C) is preferably aminomethyl propanol or arginine as the component (B) and sodium hydroxide or potassium hydroxide as the component (C), more preferably arginine as the component (B) and sodium hydroxide as the component (C).
<14> The skin cosmetic according to any one of <1> to <13>, wherein the component (D) is preferably a linear saturated alcohol having 14 to 22 of carbon atom, more preferably a linear saturated alcohol having 16 to 22 of carbon atom.
<15> The skin cosmetic according to any one of <1> to <14>, wherein the component (D) preferably contains lauryl alcohol, myristyl alcohol, cetyl alcohol, stearyl alcohol, and/or behenyl alcohol, more preferably cetyl alcohol and/or stearyl alcohol, further preferably cetyl alcohol and stearyl alcohol.
<16> The skin cosmetic according to any one of <1> to <15>, wherein the content of the component (D) in the whole composition is preferably 0.5 mass % or more, more preferably 1 mass % or more, and is preferably 3 mass % or less, more preferably 2.5 mass % or less.
<17> The skin cosmetic according to any one of <1> to <16>, wherein the total amount of the components (A) and (D), (A)+(D), in the whole composition is preferably 2 mass % or more, more preferably 4.5 mass % or more, and is preferably 10 mass % or less, more preferably 8 mass % or less.
<18> The skin cosmetic according to any one of <1> to <16>, wherein the mass ratio of the component (A) to the total amount of the components (A) and (D) [(A)+(D)], (A)/[(A)+(D)], is preferably 0.73 or more, more preferably 0.75 or more, and is preferably 0.87 or less, more preferably 0.82 or less.
<19> The skin cosmetic according to any one of <1> to <18>, wherein the component (E) is preferably a polyhydric alcohol, more preferably a dihydric or trihydric polyhydric alcohol.
<20> The skin cosmetic according to any one of <1> to <19>, wherein the component (E) has a molecular weight of preferably from 70 to 600, more preferably from 75 to 500, further preferably from 80 to 300, still further preferably from 85 to 200.
<21> The skin cosmetic according to any one of <1> to <20>, wherein the component (E) is preferably at least any one selected from the group consisting of propylene glycol, 1,3-propanediol, dipropylene glycol, 1,3-butylene glycol, glycerin, and diglycerin, more preferably at least any one selected from the group consisting of 1,3-propanediol, 1,3-butylene glycol, dipropylene glycol, and glycerin.
<22> The skin cosmetic according to any one of <1> to <21>, wherein the content of the component (E) in the whole composition is preferably 1 mass % or more, more preferably 2.5 mass % or more, and is preferably 18 mass % or less, more preferably 15 mass % or less.
<23> The skin cosmetic according to any one of <1> to <22>, wherein the mass ratio of the component (E) to the total amount of the components (A) and (D) [(A)+(D)], (E)/[(A)+(D)], is preferably 0.1 or more, more preferably 0.15 or more, further preferably 0.3 or more, and is preferably 3 or less, more preferably 2.5 or less, further preferably 1.5 or less.
<24> The skin cosmetic according to any one of <1> to <23>, wherein the content of water as the component (F) in the whole composition is preferably 50 mass % or more, more preferably 60 mass % or more, and is preferably 95 mass % or less, more preferably 90 mass % or less.
<25> The skin cosmetic according to any one of <1> to <24>, further containing (G) a nonionic surfactant, wherein the content thereof in the whole composition is preferably 0.1 mass % or more, more preferably 0.3 mass % or more, further preferably 0.5 mass % or more, and is preferably 2 mass % or less, more preferably 1.8 mass % or less, further preferably 1.5 mass % or less.
<26> The skin cosmetic according to <25>, wherein the nonionic surfactant (G) is preferably one or more selected from the group consisting of an ethylene glycol fatty acid ester, a polyethylene glycol fatty acid ester, a polyalkylene glycol alkyl ether, a polyethylene glycol hydrogenated castor oil, a propylene glycol fatty acid ester, a monoglycerin monofatty acid ester, a monoglycerin difatty acid ester, a glycerin alkyl ether, a sorbitan fatty acid ester, a fatty acid alkanolamide, and a fatty acid dialkanolamide, more preferably a polyalkylene glycol alkyl ether, further preferably a polyalkylene glycol alkyl ether having an alkyl group having 12 to 22 of carbon atom, still further preferably a polyalkylene glycol alkyl ether having an alkyl group having 12 to 18 of carbon atom.
<27> The skin cosmetic according to <25> or <26>, wherein the nonionic surfactant (G) preferably contains at least two polyalkylene glycol ethers differing in the number of carbon atom in their alkyl groups by 2 or more, more preferably contains at least two selected from the group consisting of polyethylene glycol ether of lauryl alcohol (laureth-3), polyethylene glycol ether of cetyl alcohol, and polyethylene glycol ether of stearyl alcohol, further preferably contains these 3 polyethylene glycol ethers, and still further preferably contains laureth-3 and ceteareth-20.
<28> The skin cosmetic according to any one of <25> to <27>, wherein the mass ratio of the total amount of the components (A) and (D) [(A)+(D)] to the component (G), [(A)+(D)]/G}, is preferably 3 or more, more preferably 4 or more, further preferably 5 or more, and is preferably 30 or less, more preferably 20 or less, further preferably 15 or less.
<29> The skin cosmetic according to any one of <1> to <28>, wherein the skin cosmetic has a pH of preferably from 5.5 to 9.5, more preferably from 6.0 to 9.0, further preferably from 6.5 to 8.5, at 25° C.
<30> The skin cosmetic according to any one of <1> to <29>, wherein the skin cosmetic has a bulk lamellar structure and a concentric lamellar structure.
<31> The skin cosmetic according to any one of <1> to <30>, further containing a solid fat other than the components (A) and (D), wherein the content thereof in the whole composition is preferably 10 mass % or less, more preferably 8 mass % or less, further preferably 5 mass % or less, still further preferably 3 mass % or less.
<32> The skin cosmetic according to any one of <1> to <31>, further containing an anionic surfactant, preferably one or two or more selected from the group consisting of an alkyl ether phosphate having 12 to 22 of carbon atom of polyoxyethylene or a salt thereof, sodium N-alkyloyl methyltaurine having 12 to 22 of carbon atom, and a N-acyl glutamate having 12 to 22 of carbon atom, more preferably an alkyl ether phosphate having 12 to 22 of carbon atom of polyoxyethylene or a salt thereof.
<33> The skin cosmetic according to <32>, wherein the content of the anionic surfactant in the whole composition is preferably 0.01 mass % or more, more preferably 0.1 mass % or more, further preferably 0.3 mass % or more, and is preferably 2 mass % or less, more preferably 1.8 mass % or less, further preferably 1.5 mass % or less.
<34> A method for using a skin cosmetic, containing using a skin cosmetic according to any one of <1> to <33> to the skin, preferably applying a skin cosmetic according to any one of <1> to <33> to the skin except for the scalp, more preferably any of the face, the body, limbs, and the like.
<35> A moisturizing method including applying a skin cosmetic according to any one of <1> to <33> to the skin.
<36> A method for producing a skin cosmetic according to any one of <1> to <33>, including steps 1 to 3:
step 1: the step of mixing the components (B), (C), (E), and (F) and homogeneously dissolving the mixture at 60 to 100° C. to form an aqueous phase portion,
step 2: the step of mixing the components (A) and (D) and homogeneously dissolving the mixture at 60 to 100° C. to form an oil phase portion, and
step 3: the step of homogeneously mixing the aqueous phase portion obtained in step 1 with the oil phase portion obtained in step 2 and cooling the mixture to preferably from 5 to 30° C., more preferably from 10 to 28° C., further preferably from 15 to 26° C.

### EXAMPLES

### Examples 1 to 33 and Comparative Examples 1 to 9

Skin cosmetics were produced according to the composition shown in Tables 1 to 3, and the presence or absence of bulk lamella formation in the cosmetics and dried films was observed. The skin cosmetics were evaluated for moisturizing properties (moisture-confining properties), the sustention of a moist feeling, the absence of stickiness, and uniform distribution on the skin. The results are also shown in Tables 1 to 3.

### (Production Method)

The aqueous phase components including the components (B), (C), (E), and (F) were dissolved by stirring at 70 to 80° C. to form an aqueous phase portion. Next, the oil phase components including the components (A) and (D) were dissolved by stirring at 70 to 80° C. to form an oil phase portion. The oil phase portion was added to the aqueous phase portion with stirring at 70 to 80° C., and the mixture was cooled to room temperature (25° C.) with further stirring to produce a skin cosmetic (oil-in-water emulsion cosmetic).

### (Evaluation Method)

### (1) Confirmation of Bulk Lamella Formation:

The cosmetics and dried films were evaluated for their phase states. Each dried film was prepared by spreading 1 g of each cosmetic at a thickness of 0.1 mm using an applicator and drying the resulting film at 20° C. for 24 hours in a 20% RH environment.

The samples (thickness: 25 mm) were observed under a polarization microscope (×200). A sample found shiny as a whole without detectable crystals was indicated by “Y” (bulk lamella was present), a sample found shiny as a whole but with detectable crystals was indicated by “N”, and a sample found not shiny as a whole was indicated by “N”.

### (2) Moisturizing Properties (Moisture-Confining Properties):

Each cosmetic was applied at 0.01 mL/cm² to 5C quantitative filter paper (manufactured by Toyo Roshi Kaisha, Ltd., ADVANTEC FILTER PAPER 5C) and left standing at 20° C. for 24 hours in a 20% RH environment. A portion of this filter paper was cut out and placed on a 40-mL vial (Pierce Vial CV-400, manufactured by AS ONE Corp.; the lid has an opening of 17.3 mm in diameter) to cover the top of the vial therewith. A given amount of water was added to the vial, and the resulting vial was left standing at 20° C. for 24 hours in a 20% RH environment. The amount of water decreased was measured.

The mass before storage was defined as m1; the mass after 24 hours was defined as m2; the amount of water evaporation in the case of cosmetic-unapplied filter paper was defined as W (g); and the amount of water evaporation in the case where the cosmetic of each Example or Comparative Example was applied to the filter paper was defined as S (g). The rate of suppression of water loss (%) was determined according to the following expressions:

Amount of water evaporation W(g)=Wm1−Wm2

Amount of water evaporation S(g)=Sm1−Sm2

Rate of suppression of water evaporation (%)=(W(g)−S(g))/W(g)×100

A higher numeric value represents better moisturizing properties (moisture-confining properties).

### (3) Sustention of a Moist Feeling:

Ten expert panelists uniformly applied 2 mg/cm² of each skin cosmetic on their faces after face wash and conducted sensory evaluation on a moist feeling of the skin 3 hours later according to the following criteria, and an average of the scores was determined.

4: Very moist.
3: Slightly moist.
2: Slight dryness was felt.
1: Dryness was felt.

### (4) Absence of Stickiness:

Ten expert panelists uniformly applied 2 mg/cm² of each cosmetic on their faces and conducted sensory evaluation on the absence of stickiness immediately after the application according to the following criteria, and an average of the scores was determined.

4: Very non-sticky.
3: Slightly non-sticky.
2: Slightly sticky.
1: Very sticky.

### (5) Uniform Distribution on the Skin:

Ten expert panelists uniformly applied 2 mg/cm² of each cosmetic on their faces and conducted sensory evaluation on the uniformity of the distribution of the water-soluble components on the skin after a lapse of 3 hours according to the following criteria, and an average of the scores was determined.

4: Uniformly distributed throughout the skin.
3: Slightly nonuniformly distributed on the cristae cutis.
2: Slightly nonuniformly distributed on the sulci cutis and the cristae cutis.
1: Nonuniformly distributed throughout the skin.

TABLE 1

| Component | | Example | | | | | | | | | | | | | |
|---|---|---|---|---|---|---|---|---|---|---|---|---|---|---|---|
| (mass %) | | 1 | 2 | 3 | 4 | 5 | 6 | 7 | 8 | 9 | 10 | 11 | 12 | 13 | 14 |
| A | Myristic | | | | | | | | | | | | | | 4.0 |
| | acid | | | | | | | | | | | | | | |
| | Stearic | 0.5 | 8.0 | 2.2 | 6.8 | 6.0 | 6.0 | 6.5 | 6.5 | 6.5 | 5.5 | 5.0 | 5.0 | 5.0 | |
| | acid | | | | | | | | | | | | | | |
| | Behenic | | | | | | | | | | | | | | |
| | acid | | | | | | | | | | | | | | |
| B | L- | 0.08 | 2.80 | 0.20 | 3.30 | 1.40 | 3.00 | 1.50 | 3.00 | 1.60 | 2.50 | 1.50 | 2.00 | 2.00 | 2.00 |
| | arginine | | | | | | | | | | | | | | |
| | 2-Amino- | | | | | | | | | | | | | | |
| | 2-methyl- | | | | | | | | | | | | | | |
| | 1- | | | | | | | | | | | | | | |
| | propanol | | | | | | | | | | | | | | |
| | Trieth- | | | | | | | | | | | | | | |
| | anolamine | | | | | | | | | | | | | | |
| C | Sodium | 0.01 | 0.40 | 0.03 | 0.15 | 0.18 | 0.12 | 0.20 | 0.15 | 0.15 | 0.15 | 0.12 | 0.15 | 0.15 | 0.15 |
| | hydroxide | | | | | | | | | | | | | | |
| | Potassium | | | | | | | | | | | | | | |
| | hydroxide | | | | | | | | | | | | | | |
| D | Myristyl | | | | | | | | | | | | | | 1.0 |
| | alcohol | | | | | | | | | | | | | | |
| | Stearyl | | | | | | | | | | | | | | |
| | alcohol | | | | | | | | | | | | | | |
| | Ceto- | 0.2 | 3.3 | 0.8 | 0.8 | 2.5 | 1.0 | 2.0 | 1.5 | 2.0 | 1.5 | 1.5 | 1.5 | 1.5 | |
| | stearyl | | | | | | | | | | | | | | |
| | alcohol | | | | | | | | | | | | | | |
| | Behenyl | | | | | | | | | | | | | | |
| | alcohol | | | | | | | | | | | | | | |
| E | Glycerin | 0.1 | 20.0 | 0.5 | 20.0 | 1.0 | 20.0 | 1.0 | 20.0 | 1.0 | 20.0 | 1.0 | 16.0 | 5.0 | 5.0 |
| | Dipropyl- | | | | | | | | | | | | | | |
| | ene | | | | | | | | | | | | | | |
| | glycol | | | | | | | | | | | | | | |
| | 1,3-Butyl- | | | | | | | | | | | | | | |
| | ene glycol | | | | | | | | | | | | | | |
| | Poly- | | | | | | | | | | | | | | |
| | ethylene | | | | | | | | | | | | | | |
| | glycol | | | | | | | | | | | | | | |
| F | Water | Bal- | Bal- | Bal- | Bal- | Bal- | Bal- | Bal- | Bal- | Bal- | Bal- | Bal- | Bal- | Bal- | Bal- |
| | | ance | ance | ance | ance | ance | ance | ance | ance | ance | ance | ance | ance | ance | ance |
| G | Cetear- | | | | | | | | | | | | | | |
| | eth-20 | | | | | | | | | | | | | | |
| | (HLB | | | | | | | | | | | | | | |
| | 12.0) | | | | | | | | | | | | | | |
| | Laureth-3 | | | | | | | | | | | | | | |
| | (HLB 7.0) | | | | | | | | | | | | | | |
| | Glycerin | | | | | | | | | | | | | | |
| | mono- | | | | | | | | | | | | | | |
| | stearic | | | | | | | | | | | | | | |
| | acid ester | | | | | | | | | | | | | | |
| | (HLB3.4) | | | | | | | | | | | | | | |
| | PEG-30 | | | | | | | | | | | | | | |
| | hydrogen- | | | | | | | | | | | | | | |
| | ated | | | | | | | | | | | | | | |
| | castor oil | | | | | | | | | | | | | | |
| | (HLB | | | | | | | | | | | | | | |
| | 11.0) | | | | | | | | | | | | | | |

| | | | | | | | | | | | | | | |
|---|---|---|---|---|---|---|---|---|---|---|---|---|---|---|
| Total | 100.0 | 100.0 | 100.0 | 100.0 | 100.0 | 100.0 | 100.0 | 100.0 | 100.0 | 100.0 | 100.0 | 100.0 | 100.0 | 100.0 |
| A + D | 0.7 | 11.3 | 3.0 | 7.6 | 8.5 | 7.0 | 8.5 | 8.0 | 8.5 | 7.0 | 6.5 | 6.5 | 6.5 | 5.0 |
| A/(A + D) | 0.71 | 0.71 | 0.73 | 0.89 | 0.71 | 0.86 | 0.76 | 0.81 | 0.76 | 0.79 | 0.77 | 0.77 | 0.77 | 0.80 |
| Mass ratio | | | | | | | | | | | | | | |
| B/(B + C) | 64.7 | 61.6 | 60.4 | 83.5 | 64.1 | 85.1 | 63.2 | 82.1 | 71.0 | 79.3 | 74.1 | 75.3 | 75.3 | 75.3 |
| Molar | | | | | | | | | | | | | | |
| ratio | | | | | | | | | | | | | | |
| (mol %) | | | | | | | | | | | | | | |
| (B + C)/A | 40.4 | 92.7 | 24.5 | 94.9 | 59.4 | 95.9 | 59.6 | 91.8 | 56.6 | 93.6 | 66.1 | 86.7 | 86.7 | 87.0 |
| Molar | | | | | | | | | | | | | | |
| ratio | | | | | | | | | | | | | | |
| (mol %) | | | | | | | | | | | | | | |
| (A + D)/G | — | — | — | — | — | — | — | — | — | — | — | — | — | — |
| Mass ratio | | | | | | | | | | | | | | |
| E/(A + D) | 0.14 | 1.77 | 0.17 | 2.63 | 0.12 | 2.86 | 0.12 | 2.50 | 0.12 | 2.86 | 0.15 | 2.46 | 0.77 | 1.00 |
| Mass ratio | | | | | | | | | | | | | | |
| Lamella | Y | Y | Y | Y | Y | Y | Y | Y | Y | Y | Y | Y | Y | Y |
| formation | | | | | | | | | | | | | | |
| (prepar- | | | | | | | | | | | | | | |
| ation): | | | | | | | | | | | | | | |
| bulk lamella | | | | | | | | | | | | | | |
| Lamella | Y | Y | Y | Y | Y | Y | Y | Y | Y | Y | Y | Y | Y | Y |
| formation | | | | | | | | | | | | | | |
| (coating | | | | | | | | | | | | | | |
| film): | | | | | | | | | | | | | | |
| bulk lamella | | | | | | | | | | | | | | |
| Moisturizing | 30 | 34 | 31 | 33 | 36 | 37 | 39 | 40 | 43 | 44 | 45 | 46 | 49 | 42 |
| properties | | | | | | | | | | | | | | |
| (moisture- | | | | | | | | | | | | | | |
| confining | | | | | | | | | | | | | | |
| properties) | | | | | | | | | | | | | | |
| Sustention of | 1.9 | 2.6 | 2.1 | 2.6 | 2.3 | 2.6 | 2.4 | 2.8 | 2.6 | 3.0 | 3.2 | 3.4 | 3.5 | 3.3 |
| moist feeling | | | | | | | | | | | | | | |
| Absence of | 2.4 | 1.9 | 2.5 | 2.0 | 2.6 | 2.3 | 2.9 | 2.6 | 3.0 | 2.7 | 3.2 | 2.9 | 3.3 | 3.1 |
| stickiness | | | | | | | | | | | | | | |
| Uniform | 1.7 | 1.8 | 1.8 | 1.8 | 2.1 | 2.0 | 2.2 | 2.6 | 2.6 | 2.8 | 2.6 | 3.0 | 3.2 | 3.2 |
| distribution | | | | | | | | | | | | | | |
| on skin | | | | | | | | | | | | | | |

TABLE 2

| Component | | Example | | | | | | | | | | | | | |
|---|---|---|---|---|---|---|---|---|---|---|---|---|---|---|---|
| (mass %) | | 15 | 16 | 17 | 18 | 19 | 20 | 21 | 22 | 23 | 24 | 25 | 26 | 27 | 28 |
| A | Myristic | | | | | | | | | | | | | | |
| | acid | | | | | | | | | | | | | | |
| | Stearic | 5.0 | | 5.0 | 5.0 | 5.0 | 5.0 | 5.0 | 5.0 | 5.0 | 5.0 | 5.0 | 5.0 | 5.0 | 5.0 |
| | acid | | | | | | | | | | | | | | |
| | Behenic | | 6.0 | | | | | | | | | | | | |
| | acid | | | | | | | | | | | | | | |
| B | L-arginine | 2.00 | 2.00 | | | 2.00 | 2.00 | 2.00 | 2.00 | 2.00 | 2.00 | 2.00 | 2.00 | 2.00 | 2.00 |
| | 2-Amino- | | | 1.00 | | | | | | | | | | | |
| | 2-methyl- | | | | | | | | | | | | | | |
| | 1-propanol | | | | | | | | | | | | | | |
| | Triethanol- | | | | 1.50 | | | | | | | | | | |
| | amine | | | | | | | | | | | | | | |
| C | Sodium | 0.15 | 0.15 | 0.15 | 0.15 | | 0.15 | 0.15 | 0.15 | 0.15 | 0.15 | 0.15 | 0.15 | 0.15 | 0.15 |
| | hydroxide | | | | | | | | | | | | | | |
| | Potassium | | | | | 0.20 | | | | | | | | | |
| | hydroxide | | | | | | | | | | | | | | |
| D | Myristyl | | | | | | | | | | | | | | |
| | alcohol | | | | | | | | | | | | | | |
| | Stearyl | 1.5 | | | | | | | | | | | | | |
| | alcohol | | | | | | | | | | | | | | |
| | Cetostearyl | | | 1.5 | 1.5 | 1.5 | 1.5 | 1.5 | 1.5 | 1.5 | 1.5 | 1.5 | 1.5 | 1.5 | 1.5 |
| | alcohol | | | | | | | | | | | | | | |
| | Behenyl | | 1.5 | | | | | | | | | | | | |
| | alcohol | | | | | | | | | | | | | | |
| E | Glycerin | 5.0 | 5.0 | 5.0 | 5.0 | 5.0 | 8.0 | | | | 2.5 | 5.0 | 5.0 | 5.0 | 5.0 |
| | Dipropyl- | | | | | | | 5.0 | | | 2.5 | | | | |
| | ene | | | | | | | | | | | | | | |
| | glycol | | | | | | | | | | | | | | |
| | 1,3- | | | | | | | | 5.0 | | | | | | |
| | Butylene | | | | | | | | | | | | | | |
| | glycol | | | | | | | | | | | | | | |
| | Polyethyl- | | | | | | | | | 3.0 | | | | | |
| | ene | | | | | | | | | | | | | | |
| | glycol | | | | | | | | | | | | | | |
| F | Water | Bal- | Bal- | Bal- | Bal- | Bal- | Bal- | Bal- | Bal- | Bal- | Bal- | Bal- | Bal- | Bal- | Bal- |
| | | ance | ance | ance | ance | ance | ance | ance | ance | ance | ance | ance | ance | ance | ance |
| G | Ceteareth- | | | | | | | | | | | 0.3 | 2.0 | 0.7 | 1.3 |
| | 20 | | | | | | | | | | | | | | |
| | (HLB | | | | | | | | | | | | | | |
| | 12.0) | | | | | | | | | | | | | | |
| | Laureth-3 | | | | | | | | | | | | | | |
| | (HLB 7.0) | | | | | | | | | | | | | | |
| | Glycerin | | | | | | | | | | | | | | |
| | mono- | | | | | | | | | | | | | | |
| | stearic | | | | | | | | | | | | | | |
| | acid ester | | | | | | | | | | | | | | |
| | (HLB3.4) | | | | | | | | | | | | | | |
| | PEG-30 | | | | | | | | | | | | | | |
| | hydro- | | | | | | | | | | | | | | |
| | genated | | | | | | | | | | | | | | |
| | castor oil | | | | | | | | | | | | | | |
| | (HLB | | | | | | | | | | | | | | |
| | 11.0) | | | | | | | | | | | | | | |

| | | | | | | | | | | | | | | |
|---|---|---|---|---|---|---|---|---|---|---|---|---|---|---|
| Total | 100.0 | 100.0 | 100.0 | 100.0 | 100.0 | 100.0 | 100.0 | 101.0 | 102.0 | 102.0 | 100.0 | 100.0 | 100.0 | 100.0 |
| A + D | 6.5 | 7.5 | 6.5 | 6.5 | 6.5 | 6.5 | 6.5 | 6.5 | 6.5 | 6.5 | 6.5 | 6.5 | 6.5 | 6.5 |
| A/(A + D) | 0.77 | 0.80 | 0.77 | 0.77 | 0.77 | 0.77 | 0.77 | 0.77 | 0.77 | 0.77 | 0.77 | 0.77 | 0.77 | 0.77 |
| Mass ratio | | | | | | | | | | | | | | |
| B/(B + C) | 75.3 | 75.3 | 74.9 | 72.8 | 76.3 | 75.3 | 75.3 | 75.3 | 75.3 | 75.3 | 75.3 | 75.3 | 75.3 | 75.3 |
| Molar ratio | | | | | | | | | | | | | | |
| (mol %) | | | | | | | | | | | | | | |
| (B + C)/A | 86.7 | 86.5 | 85.2 | 78.5 | 85.6 | 86.7 | 86.7 | 86.7 | 86.7 | 86.7 | 86.7 | 86.7 | 86.7 | 86.7 |
| Molar ratio | | | | | | | | | | | | | | |
| (mol %) | | | | | | | | | | | | | | |
| (A + D)/G | — | — | — | — | — | — | — | — | — | — | 26.0 | 3.3 | 9.3 | 5.0 |
| Mass ratio | | | | | | | | | | | | | | |
| E/(A + D) | 0.77 | 0.67 | 0.77 | 0.77 | 0.77 | 1.23 | 0.77 | 0.77 | 0.46 | 0.77 | 0.77 | 0.77 | 0.77 | 0.77 |
| Mass ratio | | | | | | | | | | | | | | |
| Lamella | Y | Y | Y | Y | Y | Y | Y | Y | Y | Y | Y | Y | Y | Y |
| formation | | | | | | | | | | | | | | |
| (prepar- | | | | | | | | | | | | | | |
| ation): | | | | | | | | | | | | | | |
| bulk lamella | | | | | | | | | | | | | | |
| Lamella | Y | Y | Y | Y | Y | Y | Y | Y | Y | Y | Y | Y | Y | Y |
| formation | | | | | | | | | | | | | | |
| (coating | | | | | | | | | | | | | | |
| film): | | | | | | | | | | | | | | |
| bulk lamella | | | | | | | | | | | | | | |
| Moisturizing | 48 | 51 | 44 | 42 | 47 | 48 | 48 | 46 | 42 | 50 | 52 | 51 | 56 | 54 |
| properties | | | | | | | | | | | | | | |
| (moisture- | | | | | | | | | | | | | | |
| confining | | | | | | | | | | | | | | |
| properties) | | | | | | | | | | | | | | |
| Sustention | 3.4 | 3.4 | 3.3 | 3.2 | 3.4 | 3.7 | 3.4 | 3.4 | 3.4 | 3.5 | 3.6 | 3.6 | 3.7 | 3.8 |
| of moist | | | | | | | | | | | | | | |
| feeling | | | | | | | | | | | | | | |
| Absence of | 3.4 | 3.2 | 3.3 | 3.4 | 3.4 | 3.2 | 3.4 | 3.3 | 3.3 | 3.5 | 3.5 | 3.4 | 3.6 | 3.8 |
| stickiness | | | | | | | | | | | | | | |
| Uniform | 3.3 | 3.2 | 3.3 | 3.2 | 3.4 | 3.4 | 3.4 | 3.4 | 3.4 | 3.5 | 3.5 | 3.4 | 3.8 | 3.8 |
| distribution | | | | | | | | | | | | | | |
| on skin | | | | | | | | | | | | | | |

TABLE 3

| Component | | Example | | | | | Comparative Example | | | | | | | | |
|---|---|---|---|---|---|---|---|---|---|---|---|---|---|---|---|
| (mass %) | | 29 | 30 | 31 | 32 | 33 | 1 | 2 | 3 | 4 | 5 | 6 | 7 | 8 | 9 |
| A | Myristic | | | | | | | | | | | | | | |
| | acid | | | | | | | | | | | | | | |
| | Stearic | 5.0 | 5.0 | 5.0 | 5.0 | 2.5 | | 5.0 | 5.0 | 5.0 | 0.3 | 9.0 | 5.0 | 5.0 | 3.5 |
| | acid | | | | | | | | | | | | | | |
| | Behenic | | | | | 2.5 | | | | | | | | | |
| | acid | | | | | | | | | | | | | | |
| B | L- | 2.00 | 2.00 | 2.00 | 2.00 | 1.80 | 0.50 | | 2.5 | 0.80 | 0.10 | 2.00 | 0.15 | 1.20 | 0.50 |
| | arginine | | | | | | | | | | | | | | |
| | 2- | | | | | | | | | | | | | | |
| | Amino- | | | | | | | | | | | | | | |
| | 2- | | | | | | | | | | | | | | |
| | methyl- | | | | | | | | | | | | | | |
| | 1- | | | | | | | | | | | | | | |
| | propanol | | | | | | | | | | | | | | |
| | Tri- | | | | | | | | | | | | | | |
| | ethanol- | | | | | | | | | | | | | | |
| | amine | | | | | | | | | | | | | | |
| C | Sodium | 0.15 | 0.15 | 0.15 | 0.15 | 0.15 | 0.40 | 0.60 | | 0.40 | 0.01 | 0.25 | 0.05 | 0.80 | 0.40 |
| | hydroxide | | | | | | | | | | | | | | |
| | Potassium | | | | | | | | | | | | | | |
| | hydroxide | | | | | | | | | | | | | | |
| D | Myristyl | | | | | | | | | | | | | | |
| | alcohol | | | | | | | | | | | | | | |
| | Stearyl | | | | | | | | | | | | | | |
| | alcohol | | | | | | | | | | | | | | |
| | Ceto- | 1.5 | 1.5 | 1.5 | 1.5 | 1.5 | 6.0 | 6.0 | 6.0 | | 0.4 | 10.0 | 6.0 | 6.0 | 4.0 |
| | stearyl | | | | | | | | | | | | | | |
| | alcohol | | | | | | | | | | | | | | |
| | Behenyl | | | | | | | | | | | | | | |
| | alcohol | | | | | | | | | | | | | | |
| E | Glycerin | 5.0 | 5.0 | 5.0 | 5.0 | 5.0 | 5.0 | 5.0 | 5.0 | 5.0 | 0.1 | 10.0 | 5.0 | 5.0 | 25.0 |
| | Dipro- | | | | | | | | | | | | | | |
| | pylene | | | | | | | | | | | | | | |
| | glycol | | | | | | | | | | | | | | |
| | 1,3- | | | | | | | | | | | | | | |
| | Butyl- | | | | | | | | | | | | | | |
| | ene | | | | | | | | | | | | | | |
| | glycol | | | | | | | | | | | | | | |
| | Poly- | | | | | | | | | | | | | | |
| | ethyl- | | | | | | | | | | | | | | |
| | ene | | | | | | | | | | | | | | |
| | glycol | | | | | | | | | | | | | | |
| F | Water | Bal- | Bal- | Bal- | Bal- | Bal- | Bal- | Bal- | Bal- | Bal- | Bal- | Bal- | Bal- | Bal- | Bal- |
| | | ance | ance | ance | ance | ance | ance | ance | ance | ance | ance | ance | ance | ance | ance |
| G | Cetear- | | | | 0.5 | 0.5 | | | | | | | | | |
| | eth-20 | | | | | | | | | | | | | | |
| | (HLB | | | | | | | | | | | | | | |
| | 12.0) | | | | | | | | | | | | | | |
| | Laureth- | 1.0 | | | 0.5 | 0.5 | | | | | | | | | |
| | 3 | | | | | | | | | | | | | | |
| | (HLB | | | | | | | | | | | | | | |
| | 7.0) | | | | | | | | | | | | | | |
| | Glycerin | | 1.0 | | | | | | | | | | | | |
| | mono- | | | | | | | | | | | | | | |
| | stearic | | | | | | | | | | | | | | |
| | acid ester | | | | | | | | | | | | | | |
| | (HLB3.4) | | | | | | | | | | | | | | |
| | PEG-30 | | | 1.0 | | | | | | | | | | | |
| | hydro- | | | | | | | | | | | | | | |
| | genated | | | | | | | | | | | | | | |
| | castor oil | | | | | | | | | | | | | | |
| | (HLB | | | | | | | | | | | | | | |
| | 11.0) | | | | | | | | | | | | | | |

| | | | | | | | | | | | | | | |
|---|---|---|---|---|---|---|---|---|---|---|---|---|---|---|
| Total | 100.0 | 100.0 | 100.0 | 100.0 | 100.0 | 100.0 | 100.0 | 100.0 | 100.0 | 100.0 | 100.0 | 100.0 | 100.0 | 100.0 |
| A + D | 6.5 | 6.5 | 6.5 | 6.5 | 6.5 | 6.0 | 11.0 | 11.0 | 5.0 | 0.7 | 19.0 | 11.0 | 11.0 | 7.5 |
| A/(A + D) | 0.77 | 0.77 | 0.77 | 0.77 | 0.77 | — | 0.45 | 0.45 | 1.00 | 0.43 | 0.47 | 0.45 | 0.45 | — |
| Mass ratio | | | | | | | | | | | | | | |
| B/(B + C) | 75.3 | 75.3 | 75.3 | 75.3 | 73.3 | 22.3 | — | 100.0 | 31.4 | 69.6 | 64.7 | 40.7 | 25.6 | 22.3 |
| Molar ratio | | | | | | | | | | | | | | |
| (mol %) | | | | | | | | | | | | | | |
| (B + C)/A | 86.7 | 86.7 | 86.7 | 86.7 | 87.3 | — | 85.4 | 81.7 | 83.0 | 78.1 | 56.0 | 12.0 | 153.0 | — |
| Molar ratio | | | | | | | | | | | | | | |
| (mol %) | | | | | | | | | | | | | | |
| (A + D)/G | 6.5 | 6.5 | 6.5 | 6.5 | 6. | — | — | — | — | — | — | — | — | — |
| Mass ratio | | | | | | | | | | | | | | |
| E/(A + D) | 0.77 | 0.77 | 0.77 | 0.77 | 0.77 | 0.83 | 0.45 | 0.45 | 1.00 | 0.14 | 0.53 | 0.45 | 0.45 | 3.33 |
| Mass ratio | | | | | | | | | | | | | | |
| Lamella | Y | Y | Y | Y | Y | N | N | Y | N | N | Y | N | N | N |
| formation | | | | | | | | | | | | | | |
| (prepar- | | | | | | | | | | | | | | |
| ation): | | | | | | | | | | | | | | |
| bulk | | | | | | | | | | | | | | |
| lamella | | | | | | | | | | | | | | |
| Lamella | Y | Y | Y | Y | Y | N | N | Y | N | N | Y | N | N | N |
| formation | | | | | | | | | | | | | | |
| (coating | | | | | | | | | | | | | | |
| film): | | | | | | | | | | | | | | |
| bulk | | | | | | | | | | | | | | |
| lamella | | | | | | | | | | | | | | |
| Moistur- | 55 | 57 | 52 | 58 | 62 | 6 | 23 | 30 | 11 | 10 | 30 | 19 | 20 | 22 |
| izing | | | | | | | | | | | | | | |
| properties | | | | | | | | | | | | | | |
| (moisture- | | | | | | | | | | | | | | |
| confining | | | | | | | | | | | | | | |
| properties) | | | | | | | | | | | | | | |
| Sustention | 3.7 | 3.6 | 3.5 | 3.8 | 3.9 | 1.4 | 1.5 | 1.8 | 1.4 | 1.5 | 1.6 | 1.4 | 1.8 | 1.4 |
| of moist | | | | | | | | | | | | | | |
| feeling | | | | | | | | | | | | | | |
| Absence of | 3.8 | 3.8 | 3.6 | 3.9 | 3.9 | 1.4 | 1.5 | 1.6 | 1.5 | 1.9 | 1.2 | 1.6 | 1.3 | 1.6 |
| stickiness | | | | | | | | | | | | | | |
| Uniform | 3.7 | 3.6 | 3.8 | 4.0 | 4.0 | 1.3 | 1.7 | 1.5 | 1.3 | 1.4 | 1.3 | 1.6 | 1.3 | 1.8 |
| distribution | | | | | | | | | | | | | | |
| on skin | | | | | | | | | | | | | | |

1: A skin cosmetic comprising the following components (A), (B), (C), (D), (E), and (F):
(A) 0.5 to 8 mass % of a linear saturated fatty acid having 12 to 22 of carbon atom,
(B) 0.05 to 5 mass % of an organic base,
(C) 0.01 to 2 mass % of an inorganic base,
(D) 0.1 to 5 mass % of a linear saturated alcohol having 12 to 22 of carbon atom,
(E) 0.1 to 20 mass % of a moisturizing component, and
(F) water,
wherein
the total amount of the components (A) and (D) is (A)+(D)=from 0.6 to 13 mass %, the mass ratio of the component (A) to the total amount of the components (A) and (D) [(A)+(D)] is (A)/[(A)+(D)]=from 0.7 to 0.9, the molar ratio of the component (B) to the total amount of the components (B) and (C) [(B)+(C)] is (B)/[(B)+(C)]=from 60 to 90 mol %, and the molar ratio of the total amount of the components (B) and (C) [(B)+(C)] to the component (A) is [(B)+(C)]/(A)=from 20 to 100 mol %.

2: The skin cosmetic according to claim 1, wherein the component (E) is a polyhydric alcohol.

3: The skin cosmetic according to claim 1, wherein the mass ratio of the component (E) to the total amount of the components (A) and (D) [(A)+(D)] is (E)/[(A)+(D)]=from 0.1 to 3.

4: The skin cosmetic according to claim 1, further comprising 0.1 to 2 mass % of (G) a nonionic surfactant.

5: The skin cosmetic according to claim 4, wherein the mass ratio of the total amount of the components (A) and (D) [(A)+(D)] to the component (G) is [(A)+(D)]/(G)=from 3 to 30.

6: The skin cosmetic according to claim 1, wherein component (B) is at least one member selected from the group consisting of an alkylamine having an alkyl group having 1 to 6 of carbon atom, an alkanolamine having an alkyl group having 1 to 6 of carbon atom, and a basic amino acid.

7: The skin cosmetic according to claim 1, wherein the component (C) is an alkali hydroxide.
